(19) United States
(12) Patent Application Publication
(10) Pub. No.: US 2020/0107857 A1
(43) Pub. Date: Apr. 9, 2020

(54) CHEST TUBE SHEATH

(71) Applicants: Chris Salvino, SCOTTSDALE, AZ (US); Keir Hart, Lafayette, CO (US); Mark J. Huseman, Broomfield, CO (US)

(72) Inventors: Chris Salvino, SCOTTSDALE, AZ (US); Keir Hart, Lafayette, CO (US); Mark J. Huseman, Broomfield, CO (US)

(21) Appl. No.: 16/592,728

(22) Filed: Oct. 3, 2019

Related U.S. Application Data
(60) Provisional application No. 62/742,538, filed on Oct. 8, 2018.

(51) Int. Cl. A61B 17/34

(52) U.S. Cl. CPC A61B 17/3421; A61B 2017/3427; A61B 2017/3482; A61B 2017/347; A61B 2017/3441

(57) ABSTRACT

A pliable arc-shaped chest tube that is specifically configured for being inserted and retained inside of a chest cavity provides enhanced benefits over the state of the art. The tube creates a passageway through which effluent can escape from a chest cavity, typically following trauma. The pliable arc-shaped tube can be bent to conform to a hand-held scalpel probe shaft when slid over the probe shaft. After being inserted into the chest cavity, the handheld scalpel is discarded while the chest cannula is retained or otherwise deployed within the chest cavity such that the pliable arc-shaped tube is arced towards the rib cage to enhance comfort following deployment.

### CROSS-REFERENCE TO RELATED APPLICATIONS

This application claims priority to and the benefit of U.S. provisional Patent Application No.: 62/742,538, entitled: CHEST TUBE SHEATH, filed on Oct. 8, 2018.

### FIELD OF THE INVENTION

The present embodiments are directed to a curved chest tube cannula with applications in a chest tube insertion device.

### DESCRIPTION OF RELATED ART

The lungs are surrounded by a pleural sac made up of two membranes, the visceral and parietal pleurae. The parietal pleura lines the thoracic wall, and the visceral pleura surrounds the lung. The pleural space is a potential space between these two layers of pleurae. It contains a thin layer of serous pleural fluid that provides lubrication for the pleurae and allows the layers of pleurae to smoothly slide over each other during respiration. In abnormal circumstances the pleural space can fill with air and certain types of fluids not normally present requiring drainage.

In the industrialized world, trauma is the leading cause of death in males under the age of forty. In the United States, chest injuries are responsible for one-fourth of all trauma deaths. Many of these fatalities could be prevented by early recognition of the injury followed by prompt management. Some traumatic chest injuries require quick placement of chest tubes 145 to drain out air and/or fluids (such as blood) from the chest cavity.

Several techniques are currently used to insert a chest tube 145, each of which involves a relatively lengthy manual procedure that requires knowledge and experience. The most common technique involves surgical preparation and draping at the site of the tube insertion (usually at the nipple level-fifth intercostal space, anterior to the mid-axillary line on the affected side), administering local anesthesia to the insertion site, and making a 2-4 cm vertical incision. A clamp is inserted through the incision and spread tearing muscle and tissue until a tract large enough to accept a finger is created. Next, the parietal pleura is punctured. One way is with the tip of a clamp, and the physician, on occasion, places a gloved finger into the incision to confirm the presence of a free pleural space locally. Next, the proximal end of the chest tube 145 is advanced through the incision into the pleural space. As the chest tube 145 is inserted, it is sometimes directed posteriorly and superiorly towards the apex of the lung or elsewhere in the chest cavity. Once in position, the goal is for the chest tube 145 to drain the pleural space of both air and/or fluids such as blood.

Once the chest tube 145 is appropriately in place in order to clear air and/or fluids (such as blood, infection, a transudate) from the pleural space. The tube is sutured to the skin, dressing is applied, and the tube is taped to the chest.

Insertion of a chest tube 145 using this standard technique can require more than 15 minutes to accomplish by a physician, requires extensive medical training to be performed properly and can be extremely painful as it is a difficult area to anesthetize due to the intercostal nerve that runs on the bottom of every rib. Further, while performing the procedure, the physician must attend to the patient receiving the chest tube 145 and thus is precluded from attending to other patients.

FIG. 1 depicts a prior art chest tube insertion gun 100 described in U.S. Pat. No. 7,811,293. This chest tube insertion gun 100 includes a housing 105, a handle 110 with the trigger 125, a probe tip 130 having a circular cutting tip 135 at the distal end thereof, a circular cross-sectioned cannula 140, a circular cross sectioned chest tube 145. The circular cutting tip 135 rotates outside of the distal end up to a 90° angle of rotation (rotation angle) from its neutral position before rotating back to its neutral position. The circular cutting tip 135 is also able to rotate a small negative angle from its neutral position in order to retract inside of the distal end of the probe tip 130. The rotation angle works well for the circular cross-sectioned cannula 140.

FIG. 2A illustratively depicts a prior art side view drawing of another actuator scalpel. Similar to the chest tube insertion gun 100, the actuator scalpel 200 provides a different handle system and the scalpel blade 250 that both rotates and travels outside of the probe tip 208 in a circular path. More specifically, the actuator scalpel comprises a handle body 202, a trigger 204, a probe 206, and a probe tip 208 showing the probe tip housing 212. The trigger 204 depicts finger grips 210 adapted to accommodate the fingers of a human hand (not shown). Shown for reference is the probe housing 211 and the body housing 205. In operation, the actuator scalpel 200 is gripped by an operator's (person's) palm positioned along the top of the handle body 207 with two of their fingers positioned in the finger grips 210 whereby upon squeezing the handle 204 towards the handle body 202, the scalpel 230 is made to move in a cutting motion.

FIG. 2B illustratively depicts a front isometric view of the prior art actuator scalpel 200. The scalpel blade 250 (see FIG. 2C) is arranged to extend out and beyond the probe tip housing 212 via the probe tip slot 220 when the trigger 204 is squeezed by a human hand towards the handle body 202. In this embodiment, the probe shaft 206 is oval in shape as shown, which can accommodate an oval-shaped cannula.

FIG. 2C illustratively shows the actuator scalpel 200 with a body housing (panel) 205, probe housing 211 and a probe tip housing (panel) 212 removed. A drive arm member 234 drives/moves a curved scalpel 250 by way of rotating gears 232 when the trigger 204 is actuated.

FIG. 2D shows the pathway of a single point 260 on the curved cutting edge of the prior art scalpel blade 250 while the scalpel blade 400 is deployed. The four are of a single point 260 on the cutting edge of the scalpel blade 400. Position ‘A’ is when the scalpel blade 250 is fully retracted in the probe tip housing 212; position ‘B’ is when the scalpel blade 250 has just been deployed and is pointing downwards just outside of the probe tip housing 212 via the probe tip slot 220; position ‘C’ is when the scalpel blade 250 is fully deployed and is fully extended outside of the probe tip housing 212; position ‘D’ is when the scalpel blade 250 is tipped upwards just outside of the probe tip housing 212.

FIG. 2E illustratively depicts a top view of the actuator scalpel 200 next to a prior art cannula 140. The cannula 140 is a linear tube that is arranged to slide over the probe tip 208 and cover the probe shaft 206 via a base opening 102 and a distal end opening 104. In practice, with the cannula 140 slid over the probe shaft 206, which essentially covers the probe 206, the actuator scalpel 200 is made to cut a pathway into the chest cavity of the patient whereby the cannula 140 is slid off of the probe tip 208 and thereby deployed into the chest of a patient. Accordingly, the probe 206 serves as a chest tube deployment shaft. The cannula 140 provides a drainage pathway fluid to escape the patient. Due to intercostal nerves running along the base of each rib, deploying the cannula 140 can be painful to the patient.

It is to innovations related to this subject matter that the claimed invention is generally directed.

### SUMMARY OF THE INVENTION

The present embodiments are directed to an actuating scalpel device with applications in a chest tube insertion device. The actuating scalpel device is adapted and arranged or otherwise configured to deploy a curved scalpel blade in an elliptical or circular pathway.

Certain embodiments of the present invention contemplate a curved chest cavity cannula comprising: a proximal end and a distal end; a flexible arc-shaped tube that defines at least a portion of a tunnel between the proximal end and the distal end, the flexible arc-shaped tube further defining a terminal aperture at the distal end, the flexible arc-shaped tube adapted to be deployed inside of a chest cavity between ribs and the terminal aperture capable of receiving effluent from the chest cavity; a secondary tubular portion that is not arc-shaped like the flexible arc-shaped tube, the secondary tubular portion providing a proximal aperture defining the proximal end and forming a part of the tunnel, the second tubular portion is not adapted to enter the chest cavity.

Yet other certain embodiments of the present invention contemplate a method of using a curved cannula, the method comprising: providing a flexible arc-shaped tube that defines at least a portion of a tunnel between a distal end of the curved cannula and a proximal end of the curved cannula, the tunnel defining a proximal aperture at the proximal end and a terminal aperture at the distal end; threading a chest tube deployment shaft terminating at a probe tip into the proximal aperture and through the tunnel such that at least a portion of the probe tip extends through the terminal aperture and out of the curved cannula to form a cooperating relationship with the curved cannula; pushing the chest tube deployment shaft, while in a cooperating relationship with the curved cannula, at least partially into a chest cavity via an incision accessing the chest cavity; positioning the chest tube deployment shaft in the chest cavity between ribs with the curved cannula adapted to be arced towards the ribs; holding the curved cannula in the chest cavity while removing the chest tube deployment shaft from the curved cannula so that an inner arc defined by the curved cannula is closest to the ribs, thereby completing deployment of the curved cannula in the chest cavity.

While other certain embodiments of the present invention contemplate a chest cannula comprising: a pliable arc-shaped tube specifically configured for being inserted and retained inside of a chest cavity, the tube possessing a distal aperture defined by a distal end of the arc-shaped tube, the arc-shaped tube possessing a proximal aperture at a proximal end of the arc-shaped tube, the distal aperture adapted to reside inside of the chest cavity when inserted therein, the proximal aperture is not adapted to be inserted in the chest cavity, the pliable arc-shaped tube adapted to permanently retain its arc shape when unconstrained, the pliable arc-shaped tube adapted to conform to the shape of a probe shaft used to insert the pliable arc-shaped tube inside of the chest cavity; a collar not adapted to be inserted in the chest cavity; and a stop plate butting up against the collar, the stop plate adapted to cover an incision through which the arc-shaped tube is inserted inside of the chest cavity, the chest cannula adapted to be used instead of and without cooperation of a chest tube.

### BRIEF DESCRIPTION OF THE DRAWINGS

FIG. 1 illustratively depicts a prior art drawing chest tube insertion gun;

FIG. 2A illustratively depicts a side view drawing of a prior art actuator scalpel;

FIG. 2B illustratively depicts a front isometric view drawing of the prior art actuator scalpel of FIG. 2A;

FIG. 2C illustratively depicts a drawing of the prior art actuator scalpel of FIG. 2A with many of the internal components revealed including the handle in a fully extended configuration;

FIG. 2D illustratively depict drawings of of the pathway of a single point on the curved cutting edge in various stages of scalpel blade deployment the prior art actuator scalpel of FIG. 2A;

FIG. 2E illustratively depicts a drawing of a top view of the prior art actuator scalpel of FIG. 2A next to a prior art cannula;

FIG. 3 illustratively depicts drawings of a curved cannula consistent with embodiments of the present invention;

FIGS. 4A and 4B illustratively depict drawings of a top view of the handheld actuator scalpel next a side view of the curved cannula embodiment consistent with embodiments of the present invention;

FIG. 4C illustratively depicts a front view of the curved cannula consistent with embodiments of the present invention;

FIGS. 5A-5C illustratively depict various stages of the curved cannula 300 being disposed on the probe 200 of the handheld scalpel 200 consistent with embodiments of the present invention;

FIGS. 6A and 6B illustratively depict various views of an optional embodiment of the curved cannula with drainage perforations along the shaft consistent with embodiments of the present invention;

FIG. 7 illustratively depicts a ribbed perforated curved cannula 700 consistent with embodiments of the present invention;

FIG. 8A illustratively depicts a typical location where an embodiment of the chest cannula can be deployed;

FIG. 8B illustratively depicts one embodiment of the curved cannula being inserted between ribs of a patient/subject consistent with embodiments of the present invention; and

FIG. 9 depicts an optional embodiment of a handheld actuating scalpel consistent with embodiments of the present invention.

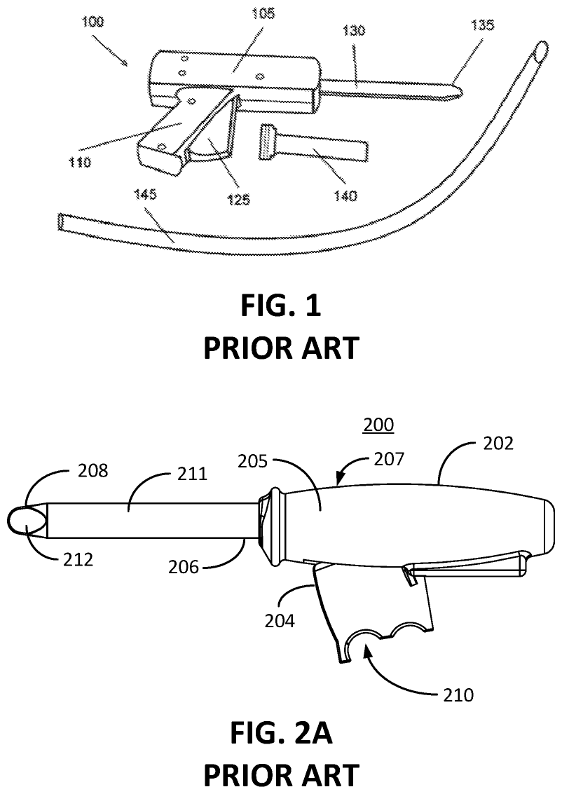

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of situations involving similar uses of a curved cannula. In what follows, similar or identical structures may be identified using identical callouts.

Described herein are embodiments of pliable arc-shaped chest tube that in some embodiments is specifically configured for being inserted and retained inside of a chest cavity thereby providing enhanced benefits over the state of the art. The chest tube creates a passageway through which effluent can escape from a chest cavity, typically following trauma. The pliable arc-shaped tube can be bent to conform to a hand-held scalpel probe shaft when slid over the probe shaft. After being inserted into the chest cavity, the handheld scalpel is discarded while the chest cannula is retained or otherwise deployed within the chest cavity such that the pliable arc-shaped tube is arced towards the rib cage to enhance comfort following deployment.

Certain embodiments herein describe a handheld actuator scalpel 200 which provides a suitable deployment device for certain curved cannula embodiments and will be used (for example) in conjunction with the curved chest tube cannula embodiment throughout this description. Deployment of the curved cannula embodiments is defined herein as the act of inserting the curved cannula inside of a chest cavity to a position in the chest cavity whereby the curved cannula can sufficiently perform its functions of draining air and/or fluid (effluent) from within the chest cavity and provide a pathway for those effluents to move outside of the chest cavity. Obviously, the curved cannula is eventually removed from the chest cavity, at which time the cannula is no longer deployed. A chest tube gun 100 also provides a suitable deployment device for the curved cannula, as can a number of other chest tube deployment devices without departing from the scope and spirit of the present invention.

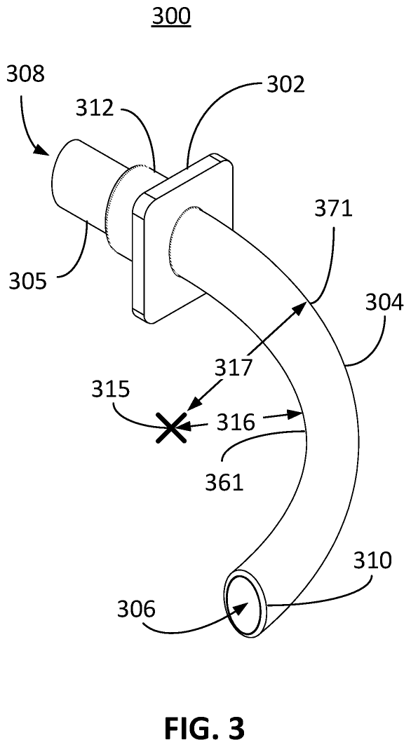

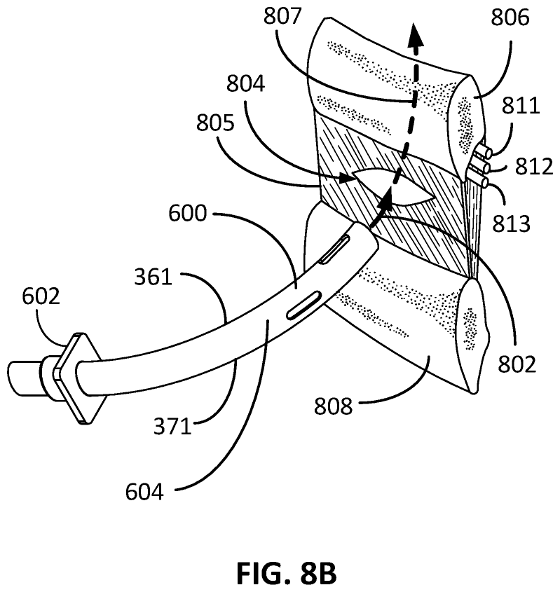

FIG. 3 illustratively depicts a curved chest tube cannula consistent with embodiments of the present invention. As shown, the curved cannula 300 generally possesses a distal end 310, a proximal end 308 and a curved tube 304. More specifically, the proximal end 308 (and more specifically the proximal aperture 308, or opening) is adapted to slide over a tip 208 of the handheld actuator scalpel 200. The distal end 310 is adapted to penetrate into the chest cavity of a recipient, which in certain embodiments is a human subject but not so limited to a human subject. A distal tube opening (or distal tube aperture) 306 in the curved cannula 300 provides an entryway into the curved cannula 300 through which fluids from the chest cavity can exit, a chest tube 145, or other devices or materials, can be threaded through and into the chest cavity. Certain embodiments envision the curved cannula 300 being pliable to conform and bend when being inserted in the chest cavity for improved maneuverability and comfort to the recipient. The curved tube 304 is essentially a pliable arc-shaped tube adapted to permanently retain its arc shape when unconstrained, such as when being held outside of a chest cavity and not engaged with a probe shaft 206. Also shown is a stop plate 302 adapted to cover an incision 804 in the subject's chest over which the curved cannula 300 can be sutured to the subject's skin (see FIGS. 8A-8B). The stop plate 302 is further adapted to help or control body fluids from leaking out of the incision 804 at the subject's chest cavity. The curved cannula 300 further comprises a rigid (essentially cannot be bent) or semi-rigid grip collar 312 that an operator can hold and manipulate with their fingers. Some embodiments contemplate the curved tube (arc-shaped tube) 304 and the collar 312 being a unitary structure delineated by a straight portion and a curved, or arc-shaped portion. Yet other embodiments contemplate the curved tube 304 and the distal portion of the tube 305 being a unitary tube which could optionally be separated by the collar 312 and the stop plate 302. Some embodiments even contemplate the distal portion of the tube 305 being curved along the same path as the curved tube portion 304 (in other words, simply a single curved tube makes up location 304 and 305). While other embodiments contemplate the collar 312 essentially further providing the purpose of being a joiner for a straight tube 305 and the curved tube 304 essentially butting up against one another to maintain a constant pathway or tunnel between the distal aperture 306 in the proximal aperture 308. In this embodiment the straight tube element 305 and the curved tubular elements 304 are fixedly attached within the collar 312 (such as by screw threads, adhesive, barbs, or other locking mechanisms known to those skilled in the mechanical arts). Certain embodiments contemplate that the distal tube opening 306 is outwardly chronically shaped as shown to provide a more comfortable (to the patient) deployment of the curved cannula 300.

The present embodiment of the curved cannula 300 contemplates being intended for deployment in a chest cavity 803 (see FIG. 8A). For reference, the inter arc 361 is defined herein, and without exception, as the arc portion of the curved cannula 300 that has the shortest radius 316 to the center of the arc 315 (i.e., the circular or elliptical center 315 of the curve that makes up the arc 316). Also for reference, the outer arc 371 is defined herein as the arc portion of the curved cannula 300 that has the longest radius 317 to the center of the arc 315. In application, when deployed in a rib cage of a human subject or otherwise, the inner arc 361 is closest to the rib cage and the outer arc 371 is closest to the patient's internal organs (e.g., heart, spine, etc.). Other embodiments envision the curved cannula 300 being deployed in other locations of a person's body that would also benefit from use of the curved cannula 300 within the scope and spirit of the present invention.

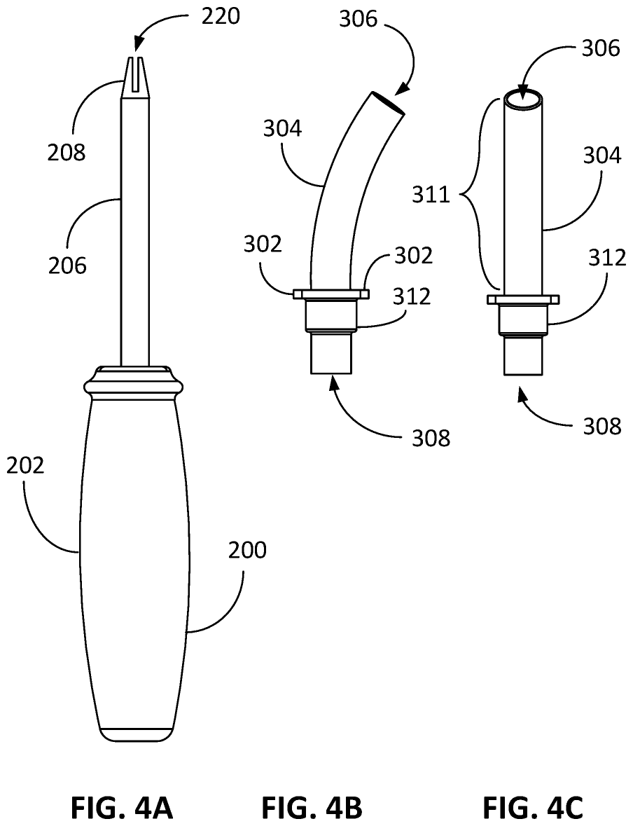

FIGS. 4A and 4B illustratively depict a top view of the handheld actuator scalpel 200 next to a side view of the curved cannula embodiment 300 consistent with embodiments of the present invention. As can be seen, the curved cannula 300 is sized to fit just below the handheld actuator scalpel distal end 208 on the probe shaft 206, which serves as a chest tube deployment shaft/structure. Also illustratively shown is the stop plate 302 abutting the collar 312. The stop plate 302 and the collar 312 can be a unitary piece of material, can be separate, can be fixedly attached together, such as via adhesive, for example. FIG. 4C illustratively depicts a front view of the curved cannula 300 revealing the distal tube aperture 306. The chest cavity tube region 311 is the portion of the curved cannula 300 that resides in a chest cavity 803 when the curved cannula 300 is deployed or otherwise inserted in a chest cavity 803. In certain embodiments, the chest cavity tube region 311 is exclusively configured to reside in a chest cavity 803.

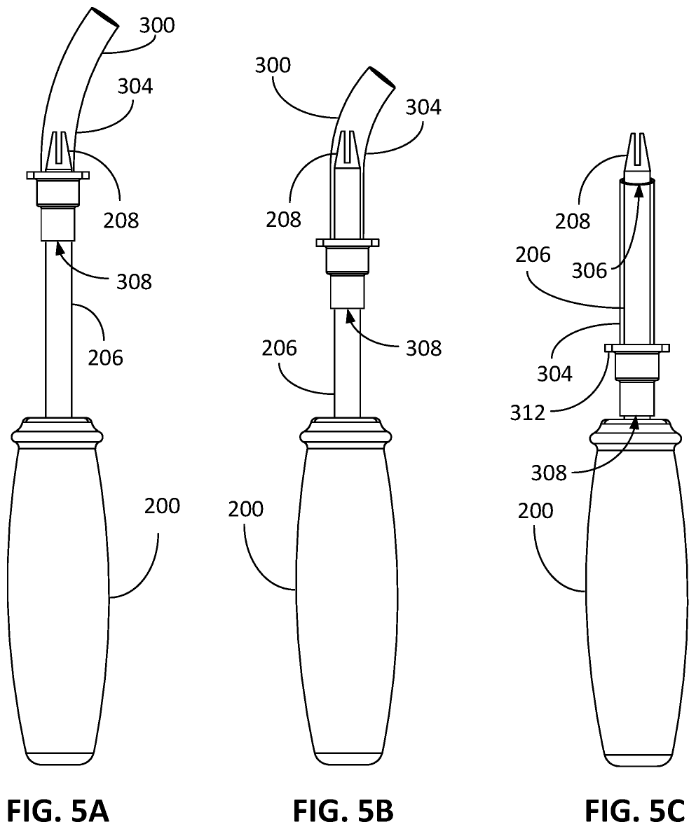

FIGS. 5A-5C illustratively depict various stages of the curved cannula 300 being disposed on the probe 200 of the handheld scalpel 200 consistent with embodiments of the present invention. As shown in FIG. 5A, the curved cannula 300 is initially placed over the probe tip 208 of the handheld scalpel 200. In this figure, only the collar 312, stop plate 302 and the distal end 308 are covering the probe shaft 206. In this embodiment, the curved tube 304 is a clear flexible polymer material (such as clear PVC vinyl tubing, Tygon PVC tubing, silicone tubing, etc.) thereby revealing the probe 206 and probe tip 208 relative to the curved tube 304. Embodiments herein envision the curved cannula 300 being manufactured from a host of optional polymer materials known to those skilled in the art. As should be readily noticed, the proximal end of the curved cannula 308 generally comprises a proximal opening, or proximal aperture, that is adapted to receive the probe tip 208.

FIG. 5B illustratively depicts the curved cannula 300 as it is partway slid over the probe tip 208 and the probe shaft 206 of the handheld scalpel 200. As shown in this figure, the curved cannula 300 embodiment is pliable (or somewhat flexible) in order to bend and thereby conform to the probe shaft 206. It should be noted that when the pliable arc-shaped tube portion 304 is not engaged with or otherwise constrained by the probe shaft 206, the pliable arc-shaped tube 304 will go back to its unconstrained state. Moreover, when the pliable arc-shaped tube portion 304 is deployed inside of a chest cavity, it is somewhat free to essentially return back to its unconstrained arc shape. Clearly, by virtue of being pliable, the arc-shaped tube portion 304 can comfortably somewhat bend and conform to the inner geometries encountered inside of a chest cavity when deployed. Certain embodiments envision the pliable arc-shaped tube portion 304 permanently retaining its arc shape when unconstrained.

FIG. 5C illustratively depicts the curved cannula 300 when it is fully slid over the probe shaft 206. As shown, the curved cannula 300 conforms to the shape of the probe shaft 206. In the embodiment shown in these figures, the curved cannula 300 is “memory shaped”. “Memory shaped” is considered that when the curved cannula 300 is deployed in a human subject, or otherwise, and then is removed from the probe shaft 206, the curved cannula 300 will be no longer straight as in FIG. 5C but rather essentially goes back (and in some embodiments, essentially immediately goes back) to its former curved shape prior to being placed on the probe shaft 206. Certain embodiments envision that the curved cannula 300 is rigid enough that when deployed in a human subject it retains (or essentially retains) the curved shape as it did prior to being deployed (and prior to being installed on a probe shaft 206). Moreover, certain embodiments envision the curved cannula 300 is rigid enough to retain, or essentially retain, the cross-sectional area of the tube 304 without collapsing and thereby shutting off the pathway created by the tube 304. Also shown more clearly in FIG. 5C, the material that defines the distal aperture 306 is slightly angled with the leading edge of the curved tube 304 being behind the probe tip 208. Certain embodiments contemplate the angle of the material that defines the distal aperture 306 being between 0° and 60° from a cross-sectional slice of the tube 304 when straightened out, such as when engaged with a probe shaft 206.

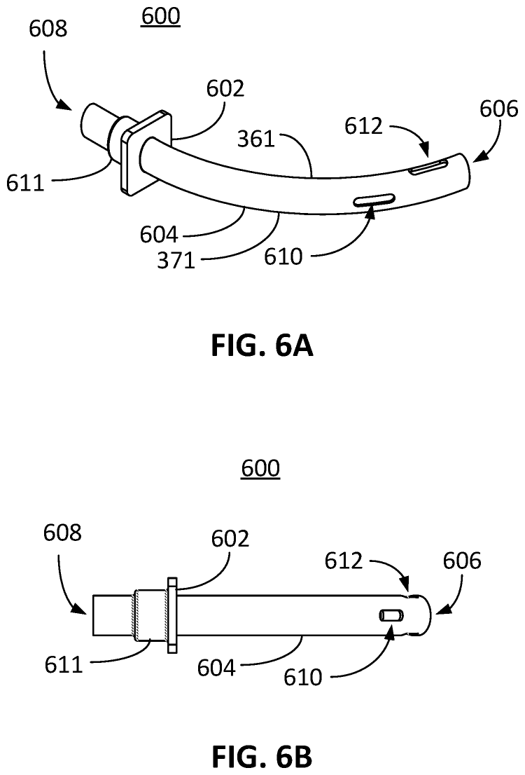

FIGS. 6A and 6B illustratively depict various views of an optional embodiment of the curved cannula with drainage perforations along the shaft consistent with embodiments of the present invention. With reference to FIG. 6A, this curved embodiment depicts a perforated curved cannula 600 with a proximal end 608 that defines a proximal aperture (not shown, but consistent with FIG. 3), a rigid or semirigid grip collar 611, a stop plate 302, a perforated curved tube 604 that terminates at a distal end 606 which defines a distal aperture (not shown). The inter arc 361 is shown for reference on the curved cannula 600. With more specificity, the perforated curved tube 604 possesses a near distal drainage perforation 610 and a far distal drainage perforation 612. As shown in FIG. 6B, the perforated curved tube 604 possesses a pair of near distal drainage perforations 610 and a pair of far distal drainage perforations 612 that are opposing and made by a hole punch device (not shown). Certain embodiments envision the hole punching device creating the drainage perforations 610 and 612 just prior (within several or a couple of minutes) to deploying the perforated curved cannula 600 and a patient or subject. Certain other embodiments envision more perforations than those shown 610 and 612. Yet other embodiments envision perforations that are not opposing one another. The perforations 610 and 612 provide enhanced drainage of body fluids within a chest cavity when the perforated curved cannula 600 is deployed in a patient or subject. The present embodiment of the curved cannula 600 contemplates being intended for deployment in a chest cavity 803 (see FIG. 8A).

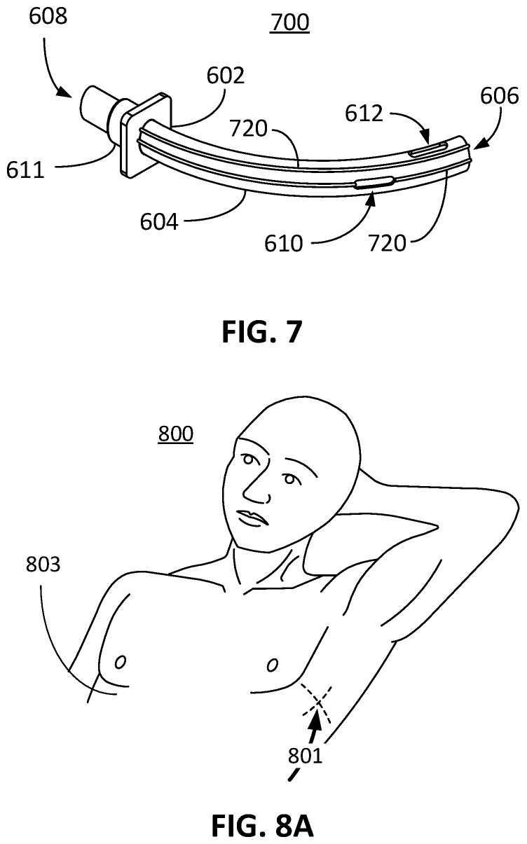

FIG. 7 illustratively depicts a ribbed perforated curved cannula 700 consistent with embodiments of the present invention. In the present embodiment, the ribbed perforated curved cannula 700 possesses all of the same features as the perforated curved cannula 600 of FIG. 6A but with the addition of ribbed features/members 720 that stand proper to the curved polymer tube 604, as shown. In the present embodiment, the ribbed members 720 run along the length of the curved tube 604 which is envisioned to be manufactured by way of extrusion techniques assuming the tube 604 is also made through extrusion techniques. The ribbed members 720 provide space for fluid to flow into the perforations 610 and 612 thereby reducing the chance of tissue collapsing around and sealing the perforations 610 and 612 when the ribbed perforated curved cannula 700 is deployed in a patient or subject. Some embodiments envision the ribbed features being concentric circles or arranged in various manners along the length of the tube 604 so long as they provide space between the perforations 610 and 612 and inner wall tissue of the patient or subject. Other embodiments contemplate a variety of shape features standing proper from the tube 604 near or at the perforations 610/612 to provide space between the inner wall tissue of a patient or subject and the tube 604 thus reducing the possibility of closing off of the perforations 610/612 (which would render the perforations 610/612 ineffective). The present embodiment of the curved cannula 700 contemplates being deployed in a chest cavity 803 (see FIG. 8A).

FIG. 8A illustratively depicts a typical location where an embodiment of the chest cannula can be deployed. As shown, the patient/subject 800 is marked with a dashed-X 801 pointing to a typical location at the fifth rib (under the armpit) where a curved chest cannula 300/600/700 can be deployed. The dashed-X 801 resides soundly in and optimal location to access the internal locations of the chest cavity 803 wherein fluid/air buildup can occur due to trauma, for example.

FIG. 8B illustratively depicts one embodiment of the curved cannula being inserted between ribs of a patient/subject consistent with embodiments of the present invention. Here, an incision 804 is made via the actuator scalpel 200 (not shown in this figure) in the intercostal muscles 805 between an upper rib 806 and a lower rib 808. Certain embodiments envision the curved cannula 600 being oval in cross-section of at least the curved polymer tube 604 in order to fit more effectively between the ribs 806 and 808 while providing greater volume of liquid/air to drain out from buildup in the chest cavity 803, typically due to trauma. The curve shaped cannula 600 provides improved comfort to the patient/subject due to the intercostal nerve 813, which runs along the bottom of each rib 806. For reference, the intercostal artery 812 and the intercostal vein 811 are shown. In practice the curved cannula 600 is fitted over the probe shaft 206 so that the probe tip 208 is unobstructed for the scalpel blade 250 to make the incision 804 (see FIGS. 5A-5C). Once the incision 804 is made, the probe tip 208 and probe shaft 206 are pressed into the chest cavity 803 up to the stop plate 602. The curved cannula 600 is positioned so that the inner-arc 361 defining the curved polymer tube 604 wraps around the upper rib 806 positioning the distal end 606 closest to the ribs 806 as shown by the path of travel of arrow 802 in the dashed final-position-arrow 807. This is defined herein as “arced towards the ribs 806” wherein the inner arc 361 is closest to the rib 806 (and rib cage 803) and the outer arc 371 is furthest away from the rib cage 803 and rib 806. The actuator scalpel 200 is pulled out by one of the operator's/surgeon's hand while the other operator's/surgeon's hand holds the curved cannula 600 in place via the grip collar 611. Once deployed, the stop plate 602 is adapted to cover the incision 804 in order to seal the incision 804. In the present embodiment, the stop plate 602 can be covered with bandage strips or sutured in place via holes (not shown) in the stop plate 602 that accommodate a needle and thread (not shown) whereby the perforated curve shaped cannula 600 is adapted to serve as a makeshift chest tube 145. Other embodiments envision using the curve shaped cannula 300 in conjunction with the chest tube 145 and feeding the chest tube 145 through the proximal aperture 308 and out the distal aperture 306 into the chest cavity 803. At this point, the curve shaped cannula 300 can be removed and the chest tube 145 sutured in place. Certain embodiments envision the curve shaped cannula 300 serving in place of a chest tube 145. In this embodiment, the flexible/pliable arc-shaped tube 600 can be sized to slide in the incision 804 just short of the spacing between the upper rib 806 and the lower rib 808. Yet other embodiments envision an oval-shaped flexible/pliable arc-shaped tube 600 so that the larger axis/length of the oval is positioned to extend towards each of the corners of the incision 804 and the shorter axis/length of the oval stretching from the upper rib 806 to the lower rib 808 when the cannula 600 is deployed.

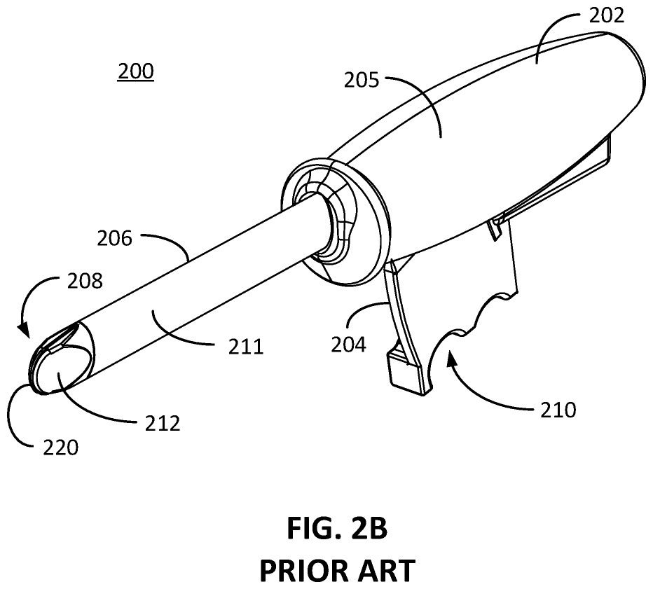

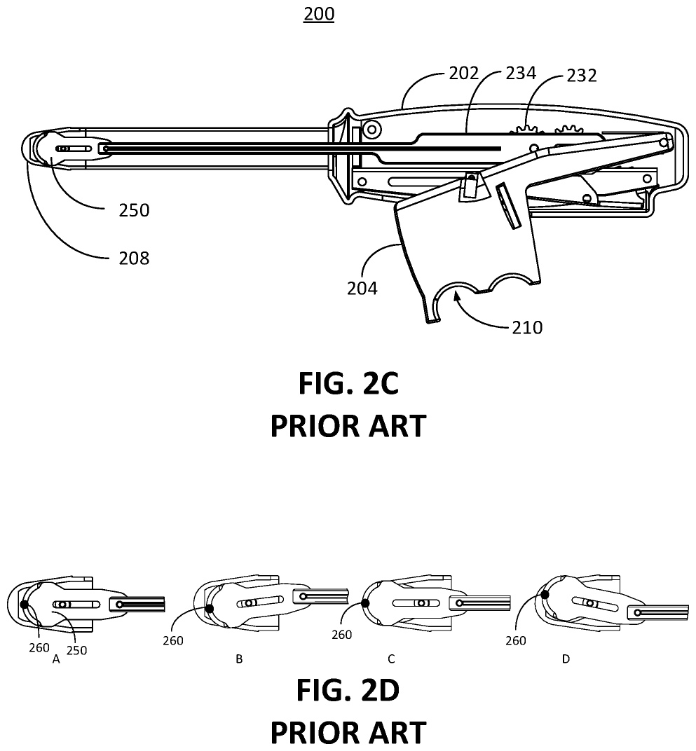

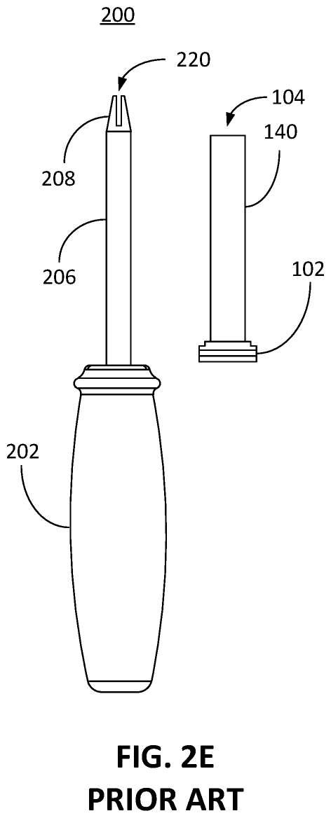

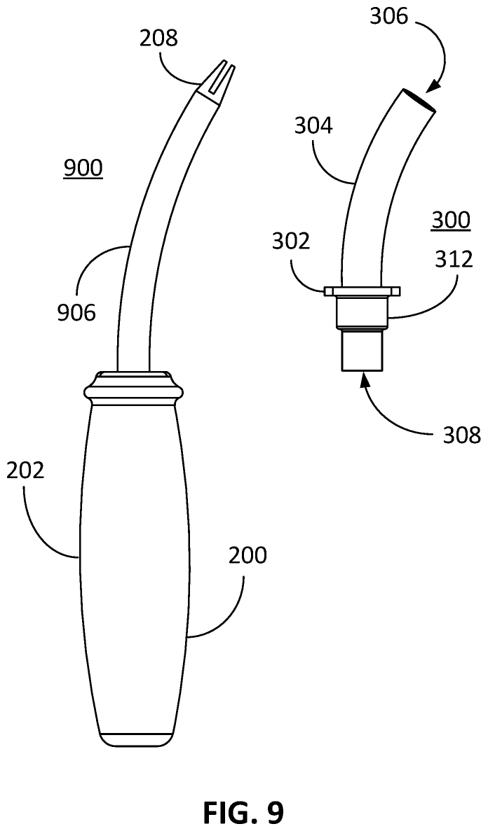

FIG. 9 depicts an optional embodiment of a handheld actuating scalpel consistent with embodiments of the present invention. In this embodiment, the handheld actuating scalpel 900 comprises an arc-shaped probe shaft 906, which can otherwise possess essentially all of the other features of the handheld actuating scalpel 200 of FIG. 2C with the exception of the drive arm member 234 which is envisioned to be modified to follow the arc-shaped probe shaft 906. Other embodiments envision other essential components shown in FIG. 2C but modified to follow the arc-shape of the probe shaft 906. The arc-shaped probe shaft 906 is depicted to conform to the shape essentially provided by the curved (arc-shaped) cannula 300.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, though a clear polymer cannula is shown in the embodiments, similar non-clear polymer cannulas could equally be used while still maintaining substantially the same functionality without departing from the scope and spirit of the present invention. Another example can include providing various other structures that may or may not include the caller 312 and/or the stop plate 302 but does comprise the curved cannula structure. Though air and fluid are envisioned as two separate compositions that can escape through the tube or tunnel created by the curved cannula embodiments, from a physics point of view air is also considered a fluid, hence, if fluid is simply used to define compositions escaping through the cannula it is reasonably considered that includes air. Yet another example can include using different kinds of perforation holes, raised elements such as ribs, or other features apparent within the scope and spirit of the present invention. Further, the terms “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

What is claimed is:

1. A curved chest cavity cannula comprising:
a proximal end and a distal end;
a flexible arc-shaped tube that defines at least a portion of a tunnel between said proximal end and said distal end, said flexible arc-shaped tube further defining a terminal aperture at said distal end, said flexible arc-shaped tube defines a chest cavity tube region, said chest cavity tube region is specifically configured to be deployed inside of a chest cavity between ribs and said terminal aperture capable of receiving effluent from said chest cavity;
a secondary tubular portion that is not arc-shaped like said flexible arc-shaped tube, said secondary tubular portion providing a proximal aperture defining said proximal end and forming a part of said tunnel, said second tubular portion is not capable of entering said chest cavity.

2. The curved chest cavity cannula of claim 1 wherein said secondary tubular portion and said flexible arc-shaped tube are comprised by a unitary tube.

3. The curved chest cavity cannula of claim 1 wherein said secondary tubular portion comprises a collar adapted to be manipulated by a human hand.

4. The curved chest cavity cannula of claim 3 wherein said collar is rigid.

5. The curved chest cavity cannula of claim 3 wherein said collar fixedly joins said secondary tubular portion with said flexible arced-shape tube.

6. The curved chest cavity cannula of claim 1 further comprising a stop plate essentially at the juncture of said flexible arc-shaped tube and said secondary tubular portion, said stop plate adapted to cover an incision in said chest cavity when said flexible arc-shaped tube is deployed in said chest cavity.

7. The curved chest cavity cannula of claim 6 wherein said stop plate is adapted to essentially seal said incision.

8. The curved chest cavity cannula of claim 1 wherein said curved chest cavity cannula is adapted to fit over a chest tube deployment shaft.

9. The curved chest cavity cannula of claim 1 wherein said curved chest cavity cannula is perforated, said perforations are adapted to enhance effusion of fluid from a person's chest cavity when said curved chest cavity cannula is deployed in said person's chest cavity.

10. The curved chest cavity cannula of claim 9 wherein said flexible arc-shaped tube possesses raised structures adapted to provide space between said perforations and tissue in said person's chest cavity.

11. The curved chest cavity cannula of claim 1 adapted to receive a flexible chest tube via said proximal aperture, said flexible chest tube adapted to be deployed in a patient's chest cavity via said terminal aperture.

12. A method of using a curved cannula, the method comprising:
providing a flexible arc-shaped tube that defines at least a portion of a tunnel between a distal end of said curved cannula and a proximal end of said curved cannula, said tunnel defining a proximal aperture at said proximal end and a terminal aperture at said distal end;
threading a chest tube deployment shaft terminating at a probe tip into said proximal aperture and through said tunnel such that at least a portion of said probe tip extends through said terminal aperture and out of said curved cannula to form a cooperating relationship with said curved cannula;
pushing said chest tube deployment shaft, while in a cooperating relationship with said curved cannula, at least partially into a chest cavity via an incision accessing said chest cavity;
positioning said chest tube deployment shaft in said chest cavity between ribs with said curved cannula arced towards said ribs;
holding said curved cannula in said chest cavity while removing said chest tube deployment shaft from said curved cannula so that an inner arc defined by said curved cannula is closest to said ribs, thereby completing deployment of said curved cannula in said chest cavity.

13. The method of claim 12 further comprising a stop plate between said proximal end and said distal end of said curved cannula wherein said curved cannula is deployed up to said stop plate.

14. The method of claim 13 wherein said stop plate covers said incision.

15. The method of claim 12 further comprising draining at least fluid and/or air from said chest cavity, said fluid and/or air entering said distal aperture and exiting said proximal aperture when said curved cannula is deployed.

16. The method of claim 15 further comprising enhancing said draining step via perforations located towards said proximal end of said flexible arc-shaped tube.

17. The method of claim 16 further comprising enhancing said draining step via protrusions that create space between said perforations and tissue interfacing said flexible arc-shaped tube when said curved cannula is deployed.

18. A chest cannula comprising:
a pliable arc-shaped tube specifically configured for being inserted and retained inside of a chest cavity, said tube possessing a distal aperture defined by a distal end of the arc-shaped tube, said arc-shaped tube possessing a proximal aperture at a proximal end of said arc-shaped tube, said distal aperture adapted to reside inside of said chest cavity when inserted therein, said proximal aperture is not adapted to be inserted in said chest cavity, said pliable arc-shaped tube adapted to permanently retain its arc shape when unconstrained, said pliable arc-shaped tube adapted to conform to the shape of a probe shaft used to insert said pliable arc-shaped tube inside of said chest cavity;
a collar not adapted to be inserted in said chest cavity; and
a stop plate butting up against said collar, said stop plate adapted to cover an incision through which said arc-shaped tube is inserted inside of said chest cavity, said chest cannula adapted to be used instead of and without cooperation of a chest tube.

19. The chest cannula of claim 18 further comprising perforations located in said arc-shaped tube, said perforations are adapted to enhance effusion of fluid from said chest cavity when said chest cannula is deployed in said chest cavity.

20. The curved chest cavity cannula of claim 19 wherein said arc-shaped tube possesses raised ribs that run lengthwise along said arc-shaped tube, said raised ribs are adapted to provide space between said perforations and tissue in said chest cavity.
